(19) United States
(12) Patent Application Publication
(10) Pub. No.: US 2011/0280924 A1
(43) Pub. Date: Nov. 17, 2011

(54) Biodegradable filler for restoration of alveolar bones

(75) Inventors: Chien-Hsing Lin, Rende Township (TW); Yu-Te Lin, Anding Township (TW); Hsiang-Yin Lu, Pingtung City (TW); Chin-Fu Wang, Xindian City (TW); Dar-Jen Hsieh, Luzhu Township (TW)

(73) Assignee: SunMax Biotechnology Co., Ltd., Tainan County (TW)

(21) Appl. No.: 12/801,983

(22) Filed: Jul. 7, 2010

(30) Foreign Application Priority Data
May 12, 2010 (TW) 099115124

(51) Int. Cl. A61K 9/70; A61P 19/00; A61K 33/42; A61K 38/39; A61K 9/14

(52) U.S. Cl. 424/443; 514/16.7; 424/489; 424/602

(57) ABSTRACT

A biodegradable filler for restoration of alveolar bones is disclosed, which includes: first cross-linked collagen fibers prepared from reacting Non-crosslinked collagen fibers with a cross-linking agent; and supporting particles which are biomedical ceramic particles, bioactive glass, or a combination thereof, and distributed among the first cross-linked collagen fibers.

### BACKGROUND OF THE INVENTION

1. Field of the Invention

The present invention relates to a biodegradable filler for restoration of alveolar bones and, more particularly, to a biodegradable filler with low degradation rate and good flexibility for restoration of alveolar bones.

2. Description of Related Art

Previously, in the case that a tooth of a patient can not maintain its original functions owing to fracture by external force, dental caries, periodontosis, or pathological changes around roots of teeth, the tooth has to be removed and then a cavity resulted thereby is filled with sterilized gauze so as to stop effusion of the blood and to restore the wound. However, the gauze has drawbacks of being non-absorbable, easily embedded with food residues, and single purpose, i.e., to stop effusion of the blood. As a result, infection of wounds easily occurs and recovery thereof needs longer time.

In recent years, tooth fillers made of collagen have been realized. Such tooth fillers only consist of collagen and they are totally bio-absorbable and have porous structure for support, provision of cell growth space, and absorption of blood. Although such products are helpful for restoration of alveolar bones, the products implanted in the hole of alveolar bones will be completely absorbed by the patient within two weeks or less since the collagen used in the products is not cross-linked.

However, in this short period of time, the patient's bone cells can not reconstruct appropriate alveolar bones such that the newly-formed alveolar bones can not return to the original size. In addition, because there is no tooth requiring support around the newly-formed alveolar bones, they are absorbed as time passes by, that is, alveolar atrophy. Accordingly, the absorbance of the alveolar bones aggravates the reduction of their height and width, leading to an incline of the normal teeth neighboring the extracted tooth.

Whether the amount of the alveolar bones in the defect region posterior to tooth extraction is sufficient or not will influence the stability of the normal teeth neighboring the extracted tooth as well as the success of the dental implant surgery. Since the dental implant requires sufficient alveolar bones for fixation, regeneration of alveolar bones becomes a necessary process prior to the dental implant surgery.

Therefore, there is an urgent need to provide a biodegradable filler for restoration of alveolar bones. When the biodegradable filler is loaded in the hole of the defect region, it can be attached by bone cells for growth. Besides, the degradation rate of the biodegradable filler approximates to the growth rate of the bone cells. Thus, the newly-formed alveolar bones are similar to original alveolar bones so as to reduce the possibility of alveolar atrophy and to prevent the inclines of the neighboring normal teeth.

### SUMMARY OF THE INVENTION

In view of the abovementioned shortcomings, the present invention uses biomedical ceramic particles or bioactive glass with high biocompatibility as well as collagen fibers chemically cross-linked to form a filler for restoration of alveolar bones. The chemically cross-linked collagen fibers can delay the degradation rate of the whole scaffold close to the growth rate of the bone cells attached thereon. Thus, the bone cells reproduce and complement the reduced volume of the scaffold during its degradation in a sufficient period of time. Therefore, it is advantageous for alveolar bones to be restored to a flat condition without defects or atrophy. In addition, the filler of the present invention is sufficiently flexible to be formed in various shapes based on the wound.

Accordingly, the present invention provides a biodegradable filler for restoration of alveolar bones, which contains: first cross-linked collagen fibers prepared by reacting non-crosslinked collagen fibers with a cross-linking agent; and supporting particles which are biomedical ceramic particles, bioactive glass, or a combination thereof, and distributed among the first cross-linked collagen fibers.

In one aspect of the present invention, the biodegradable filler for restoration of alveolar bones can further contain: second cross-linked collagen fibers, wherein the first cross-linked collagen fibers and the supporting particles can be formed in a first predetermined shape, and the second cross-linked collagen fibers totally encompass the first predetermined shape so as to form a second predetermined shape. The second predetermined shape can be a bullet-shaped column, a bulbous-headed cone, or a flat-headed cone. A thickness of the second cross-linked fibers can be in a range from 0.1 to 0.3 mm. The first cross-linked collagen fibers and the supporting particles can be uniformly distributed in the first predetermined shape.

In another aspect of the present invention, the biodegradable filler for restoration of alveolar bones can further contain: the second cross-linked fibers, wherein the first cross-linked fibers and the supporting particles can be formed in a first predetermined shape, and the second cross-linked fibers can be arranged on a surface of the first predetermined shape. A ratio of a thickness of the second cross-linked fibers to a height of the first predetermined shape can be in a range from 1:5 to 3:2. In addition, the first cross-linked collagen fibers and the supporting particles can be uniformly distributed in the first predetermined shape.

The second cross-linked fibers can be different from or the same as the first cross-linked fibers. For example, the cross-linking degree, concentration, type, etc. of collagen fibers used in the first cross-linked fibers can be respectively different from or the same as those used in the second cross-linked fibers, and thus this manner can be helpful to regulate the degradation rate of the filler of the present invention.

In the biodegradable filler for restoration of alveolar bones of the present invention, a particle size of the bioactive glass can be in a range from 100 to 700 μm, but preferably in a range from 150 to 600 μm, for example, 200, 250, 300, 350, 400, 550 μm, etc. A particle size of the biomedical ceramic particles can be in a range from 0.05 to 6.0 mm, but preferably in a range from 0.5 to 1.0 mm, for example, 0.7, 0.9 mm, etc. A pore size of the biomedical ceramic particles can be in a range from 50 to 600 but preferably in a range from 75 to 150 μm, for example, 100, 125 μm, etc. In general, hydroxyapatite (HAP), β-tri-calcium phosphate (β-TCP), HAP/β-TCP composite, or a combination thereof can be used as the biomedical ceramic particles. In the HAP/β-TCP composite, a weight ratio of HAP to β-TCP can be in a range from 1:1 to 3:1, for example, 3:2, 7:3, 2:1, 7:4, etc.

In the biodegradable filler for restoration of alveolar bones of the present invention, a weight ratio of the first cross-linked collagen fibers to the supporting particles can be in a range from 1:1 to 1:4, for example, 5:8, 2:5, 3:7, and so on.

In the biodegradable filler for restoration of alveolar bones of the present invention, the first predetermined shape can be a bullet-shaped column, a bulbous-headed cone, or a flat-headed cone. The non-cross-linked collagen fibers can be type I collagen, type II collagen, type III collagen, or a combination thereof. The cross-linking agent can be an aldehyde-based cross-linking agent, a carbodiimide-based cross-linking agent, or a combination thereof. For example, the use of the aldehyde-based cross-linking agents such as formaldehyde, acetaldehyde, propionaldehyde, valeraldehyde, glyoxal, and glutaraldehyde, or the combination of the carbodiimide-based cross-linking agents such as 1-ethyl-3-(3-dimethylaminopropyl)-carbodiimide (EDC) and N-hydroxysuccinimide (NHS) can achieve the purpose of cross-linking collagen.

Among the biomedical ceramic particles used in the present invention, β-tricalcium phosphates (β-TCP) and hydroxyapatite (HA) can play a role of a supporting scaffold for the growth of bone cells because they are porous and not easily absorbed by human bodies. Furthermore, the biomedical ceramics particles are dispersed in the collagen to form structural space of support. The collagen fibers are used for fixation and to prevent leakage of the biomedical ceramic particles. Hence, when the filler is applied in the alveolar defects, it is advantageous to achieve guided bone regeneration (GBR).

Since various patients suffer different degrees of alveolar bone defects, the time consumptions of the restorations are also unlike. However, the restoration of the alveolar bones takes about 3 to 6 months. Even though conventional collagen fillers can be used to stop effusion of blood and for restoration of alveolar bones, they will be completely absorbed within about 3 to 4 weeks. However, the filler of the present invention can act as a support for attachment of bone cells when being loaded in the hole of the alveolar bones of the patient. Moreover, the filler degrades slowly owing to the collagen fibers used in the filler being chemically cross-linked. Newly-formed bone tissues form as the filler gradually degrades. Thus, the filler can prevent alveolar bone defects and atrophy resulting from conventional collagen fillers that degrade too fast. Rapid degradation will cause insufficient support and time for bone cell growth.

In conclusion, the filler of the present invention includes the following advantages: (1) having macroporous and microporous structure, and reticular structure with highly internal connection which benefits cell growth and transportation of nutrients and metabolites, (2) being biocompatible and absorbable and having a degradation rate regulated according to absorbance and the growth rate of the newly-formed bone tissues, (3) having an appropriate structure of a porous scaffold beneficial for the attachment, proliferation, and differentiation of the bone cells, and (4) having physical properties coinciding with those of the tissue to receive the fill.

Other objects, advantages, and novel features of the invention will become more apparent from the following detailed description when taken in conjunction with the accompanying drawings.

### BRIEF DESCRIPTION OF THE DRAWINGS

FIGS. 1A to 1C show a flow chart for manufacturing a biodegradable filler for restoration of alveolar bones in Example 1 of the present invention;

FIG. 2 is a scanning electron microscopy (SEM) picture of the filler in Example 1 of the present invention;

FIGS. 3A to 3D show a flow chart for manufacturing a biodegradable filler for restoration of alveolar bones in Example 2 of the present invention;

FIG. 4 is a scanning electron microscopy (SEM) picture of the filler in Example 2 of the present invention;

FIG. 5 is a scanning electron microscopy (SEM) picture of a filler in Example 3 of the present invention;

FIGS. 6A to 6G show a flow chart for manufacturing a biodegradable filler for restoration of alveolar bones in Example 4 of the present invention;

FIG. 7 is a scanning electron microscopy (SEM) picture of the filler in Example 4 of the present invention;

FIG. 8 is a perspective view of a filler in Example 5 of the present invention;

FIG. 9 is a perspective view of a filler in Example 6 of the present invention;

FIG. 10 is a perspective view of a filler in Example 7 of the present invention;

FIG. 11 is a perspective view of a filler in Example 8 of the present invention;

FIG. 12 is a perspective view of a filler in Example 9 of the present invention;

FIG. 13 is a perspective view of a filler in Example 10 of the present invention; and

FIG. 14 is an SEM picture of freeze-dried collagen fibers which are cross-linked collagen fibers in concentration of 30±0.2 mg/ml prior to being freeze-dried in the present invention.

### DETAILED DESCRIPTION OF THE PREFERRED EMBODIMENT

In the present invention, in regard to preparation of cross-linked collagen fibers, the reactive concentration of aldehyde cross-linking agents such as glutaraldehyde, formaldehyde, and glyoxal can be in a range from 0.001-0.007%, but preferably is 0.003%. If the combination of carbodiimide-based cross-linking agents such as EDC and NHS is used as a cross-linking agent, the reactive concentration of EDC can be in a range from 0.001-0.010%, but preferably is 0.004%; the reactive concentration of NHS can be in a range from 0.0010-0.0025%, but preferably is 0.0016%.

The prepared cross-linked collagen fibers can be diluted by a phosphate buffer for cross-linked collagen fiber paste with concentration ranging from 10 to 40 mg/ml, but preferably with concentration of 30±0.2 mg/ml. Furthermore, concentration of the phosphate buffer used for dilution may not be limited in 0.2 M as long as a mixture of collagen and the phosphate buffer can be stabilized in a pH value of 7.0±0.2.

In the present invention, bioactive glass and biomedical ceramic particles can be used as the supporting particles, and useful biomedical ceramic particles are, for example, hydroxyapatite (HAP), β-tricalcium phosphate (β-TCP), HAP/β-TCP composite, or a combination thereof.

In the HAP/β-TCP composite, a ratio of HAP to β-TCP can be 60-70%:40-30% by weight. In a case that the HAP/β-TCP composite is used as the supporting particles, a ratio of the cross-linked collagen fibers to the HAP/β-TCP composite can be 20-50%:50-80% by weight, but preferably 30%:70%. In another case that the bioactive glass or the HAP alone is used as the supporting particles, a ratio of the cross-linked collagen fibers to the bioactive glass or HAP can be 20-50%:50-80% by weight, but preferably 30%:70%. In another case that the bioactive glass and the HAP/β-TCP composite are used together as the supporting particles, a ratio of the cross-linked collagen fibers to the HAP/β-TCP composite to the bioactive glass can be 20-50%:25-40%:25-40% by weight, but preferably 30%:35%:35%.

In the supporting particles mentioned above, a particle size of β-TCP can be 0.5-2.0 mm. For example, β-TCP with a particle size of 1.0-1.5 mm can be used. A particle size of HAP can be 75-150 μm. For example, HAP with a particle size of 100-125 μm can be used. A particle size of the bioactive glass can be 100-700 μm. For example, the bioactive glass with a particle size of 200-500 μm, 250-400 μm, or 450-700 μm can be used.

When the biodegradable filler for restoration of alveolar bones in the present invention is prepared, a use of a shaping mold is required. There is a hollow room inside the shaping mold. The shape of the hollow room is not limited, and can be determined by the demand of the shape of the filler. For example, the hollow room can be in a form of a bullet-shaped column, a bulbous-headed cone, or a flat-headed cone. The shaping mold can be made of any material, but the material should not vary in a temperature ranging from −60-50° C. For example, iron, stainless steel, and aluminum can be the material.

Because of the specific embodiments illustrating the practice of the present invention, a person having ordinary skill in the art can easily understand other advantages and efficiency of the present invention through the content disclosed therein. The present invention can also be practiced or applied by other variant embodiments. Many other possible modifications and variations of any detail in the present specification based on different outlooks and applications can be made without departing from the spirit of the invention.

### Preparation of the Cross-Linked Collagen Fibers

Atelocollagen with the concentration of 3.0±0.5 mg/mL was added in a phosphate buffer (0.2 M). The mixture was adjusted to a pH value of 7.0±0.2 and stirred for 4 hours. To the mixture, glutaraldehyde was added until its final concentration became 0.003%. Alternatively, the combination of EDC with final concentration of 0.004% and NHS with final concentration of 0.0016% also could be used as a cross-linking agent. The resultant mixture was adjusted to a pH value of 5.5±0.2 and stirred at 35±5° C. for 16 hours to achieve chemical cross-linking.

Posterior to chemical cross-linking, resultant cross-linked collagen fibers were homogenized at 10000±200 rpm for 10±2 minutes, and then centrifuged at 14000 G for 1 hour. The pellet, i.e. the cross-linked collagen fibers was collected. The resultant cross-linked collagen fibers were in concentration ranging from 65.0 to 100.0 mg/ml.

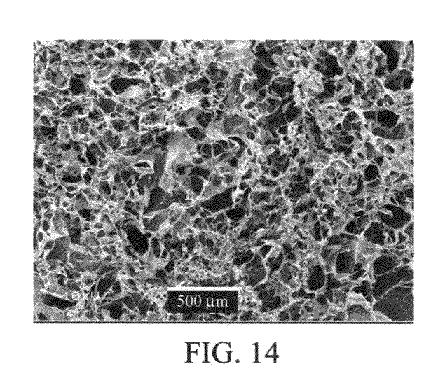

The centrifuged cross-linked collagen fibers were diluted with the phosphate buffer (0.2 M, pH 7.0±0.2) to form cross-linked collagen fiber paste with the collagen concentration of 30±0.2 mg/ml. The resultant cross-linked collagen fiber paste was freeze-dried and observed by SEM in regard to its surface and pore size. The result is shown in FIG. 14, and the pore size ranges from 50 to 400 μm.

### Example 1

An HAP/β-TCP composite was used as supporting particles. The particle size of β-TCP ranged from 0.5 to 2.0 mm, and that of HAP ranged from 0.075 to 0.150 mm. A ratio of β-TCP to HAP was 60:40% by weight.

The HAP/β-TCP composite was added into the cross-linked collagen fiber paste with 30±0.2 mg/mL collagen. A ratio of the cross-linked collagen fiber paste to the HAP/β-TCP composite was 30%:70% by weight.

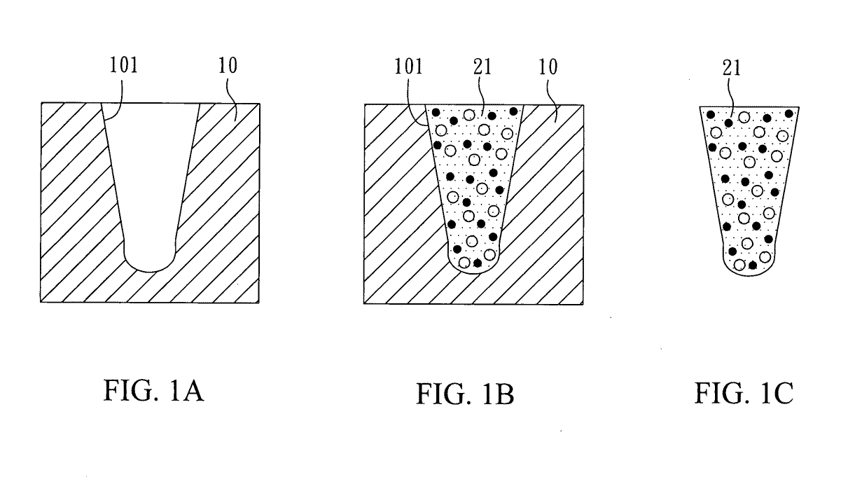

With reference to FIGS. 1A to 1C, there is shown a method for manufacturing a biodegradable filler for restoration of alveolar bones in the present invention. First, as shown in FIG. 1A, a shaping mold 10 was prepared. The shaping mold 10 had a shaping hollow 101. An internal diameter of the shaping hollow 101 reduced from the opening to the inside, and thus the shaping hollow 101 was similar to a horn. In addition, the shaping hollow 101 had an arc bottom. Therefore, a filler deposited in the shaping hollow was in a form of a bulbous-headed cone. The opening diameter of the shaping hollow 101 could range from 6.0 to 10.0 mm, and the bottom radius thereof could range from 3.0 to 5.0 mm. Besides, the depth of the shaping hollow 101 could range from 10 to 25 mm. The shaping mold 10 was made of stainless steel that could keep the mold stable during freeze-drying.

Condition of Freeze-Drying:

| | |
|---|---|
| Vacuum: 0.75 torr | |
| Freeze: −40° C. | 4 hours |
| Primary Drying: 0° C. | 72 hours |
| Second Drying: 30° C. | 24 hours |

Subsequently, as shown in FIG. 1B, the mixture 21 containing the cross-linked collagen fiber paste and the HAP/β-TCP composite was slowly loaded in the shaping mold 10, and thus the situation that bubbles were embedded in the shaping mold 10 could be prevented during loading. After loading, the shaping mold 10 together with the mixture 21 was freeze-dried in the condition as mentioned above.

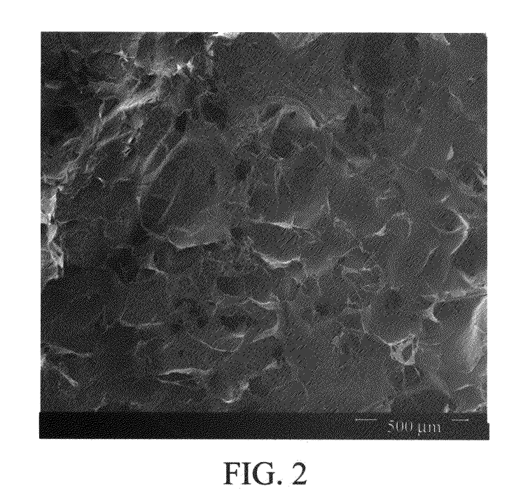

After freeze-drying was completed, the filler as shown in 1C was removed from the shaping mold 10, and observed by SEM in regard to its surface and pore size. The result is shown in FIG. 2, and the pore size ranges from 200 to 500 μm.

### Examples 2 and 3

HAP (Example 2) or bioactive glass (Example 3) was used as supporting particles. The particle size of HAP ranged from 0.075 to 0.15 mm, and that of HAP ranged from 150 to 600 μm.

The HAP or bioactive glass was added into the cross-linked collagen fiber paste with 30±0.2 mg/mL collagen. A ratio of the cross-linked collagen fiber paste to the HAP or bioactive glass was 40%:60% by weight.

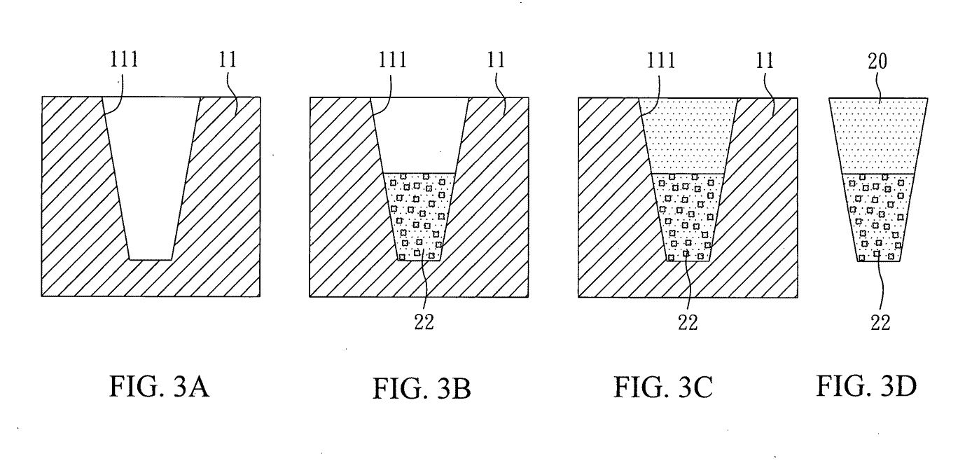

With reference to FIGS. 3A to 3D, there is shown a method for manufacturing a biodegradable filler for restoration of alveolar bones in the present invention. First, as shown in FIG. 3A, a shaping mold 11 was prepared. The shaping mold 11 had a shaping hollow 111. An internal diameter of the shaping hollow 111 reduced from the opening to the inside. In addition, the shaping hollow 111 had a flat bottom. Therefore, a filler formed in the shaping hollow 111 was in a form of a flat-headed cone. The shaping mold 11 was made of iron that could keep the mold stable during freeze-drying.

Subsequently, as shown in FIG. 3B, the mixture 22 containing the cross-linked collagen fiber paste and the bioactive glass was slowly loaded in the shaping mold 11 until ½ to ⅔ volume of the shaping mold 11 was filled. As shown in FIG. 3C, the cross-linked collagen fiber paste (30 mg/ml) was loaded in the shaping mold 11 and it covered on the mixture 22 until the shaping mold 11 was full.

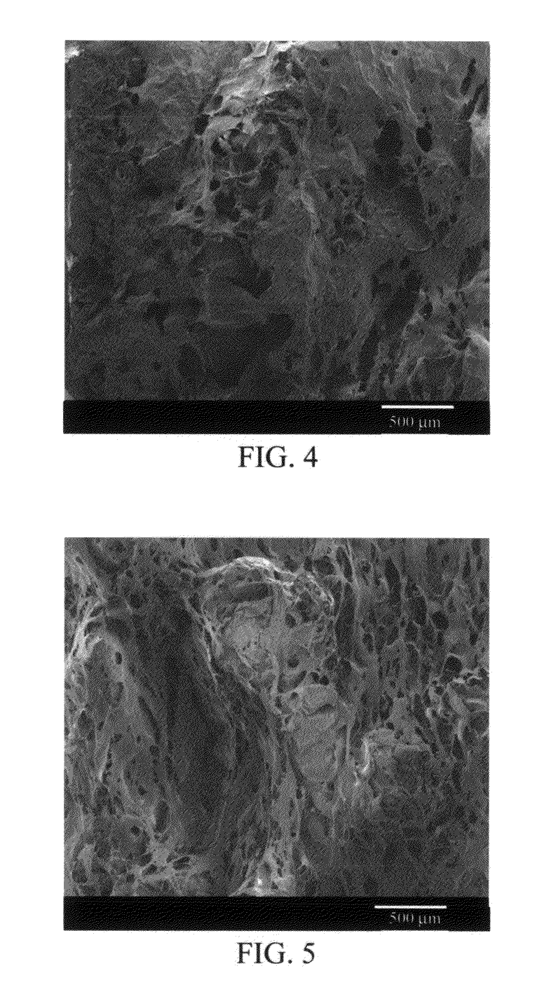

After loading, the shaping mold 11 together with the mixture 22 and cross-linked collagen fiber paste (30 mg/ml) 20 was freeze-dried in the condition as mentioned in Example 1. After freeze-drying was completed, the filler as shown in FIG. 3D was removed from the shaping mold 11, and observed by SEM in regard to its surface and pore size. The results of Examples 2 and 3 are shown in FIGS. 2 and 3, respectively. The pore size in FIG. 4 ranges from 200 to 500 μm, and that in FIG. 5 ranges from 50 to 300 μm.

### Example 4

The HAP/β-TCP composite and the bioactive glass were used as supporting particles. The particle size of the HAP/β-TCP composite ranged from 0.5 to 1.0 mm, and that of the bioactive glass ranged from 150 to 600

The HAP/β-TCP composite and the bioactive glass were added into the cross-linked collagen fiber paste with 30±0.2 mg/mL collagen. A ratio of the cross-linked collagen fiber paste to the HAP/(3-TCP composite to the bioactive glass was 30%:35%:35% by weight.

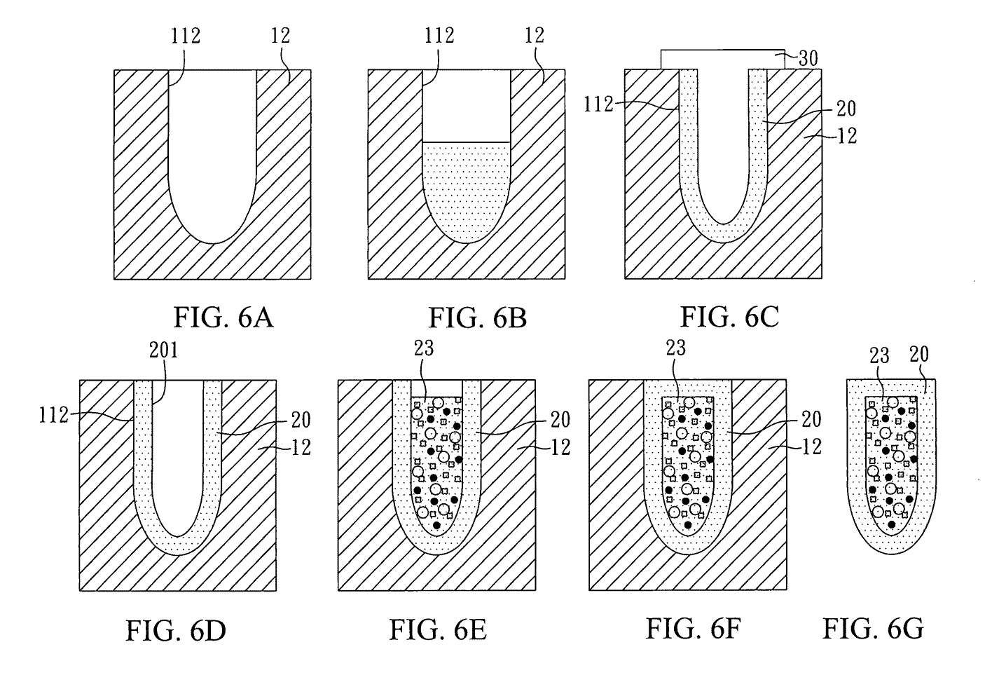

With reference to FIGS. 6A to 6G, there is shown a method for manufacturing a biodegradable filler for restoration of alveolar bones in the present invention. First, as shown in FIG. 6A, a shaping mold 12 was prepared. The shaping mold 12 had a shaping hollow 112. An internal diameter of the shaping hollow 112 was approximately identical from the opening to the inside, but it reduced from the inside to the bottom. Therefore, a filler deposited in the shaping hollow 112 was in a form of a bullet-shaped column. The shaping mold 12 was made of aluminum that could keep the mold stable during freeze-drying.

Subsequently, as shown in FIG. 6B, the cross-linked collagen fiber paste (30 mg/ml) 20 was slowly loaded in the shaping mold 12 until ⅓ volume of the shaping mold 12 was filled. As shown in FIG. 6C, a hollow-forming mold 30 was inserted into the center of the cross-linked collagen fiber paste 20 in the shaping mold 12. The shaping mold 12 together with the cross-linked collagen fiber paste 20 was frozen for 4±0.5 hours at −10 to −40° C. However, the freezing time is not limited to the abovementioned time because the freezing time of 20 hours still can achieve the same result. Then, as shown in FIG. 6D, the hollow-forming mold 30 was taken out and a center hole 201 was formed in the cross-linked collagen. As shown in FIG. 6E, the mixture 23 containing the cross-linked collagen fiber paste, the HAP/β-TCP composite, and the bioactive glass was loaded in the center hole 201, but the center hole 201 was not full of the mixture 23. Then, as shown in FIG. 6F, the cross-linked collagen fiber paste 20 was loaded in the center hole 201 and it covered on the mixture 23 until the center hole 201 was full.

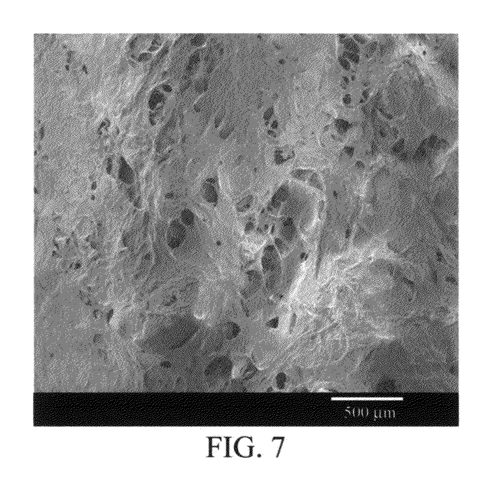

After loading, the shaping mold 12 together with the mixture 23 was freeze-dried in the condition as mentioned in Example 1. After freeze-drying was completed, the filler as shown in FIG. 6G was taken out the shaping mold 12, and observed by SEM in regard to its surface and pore size. The result is shown in FIG. 7. The pore size ranges from 200 to 500 μm.

### Examples 5 and 6

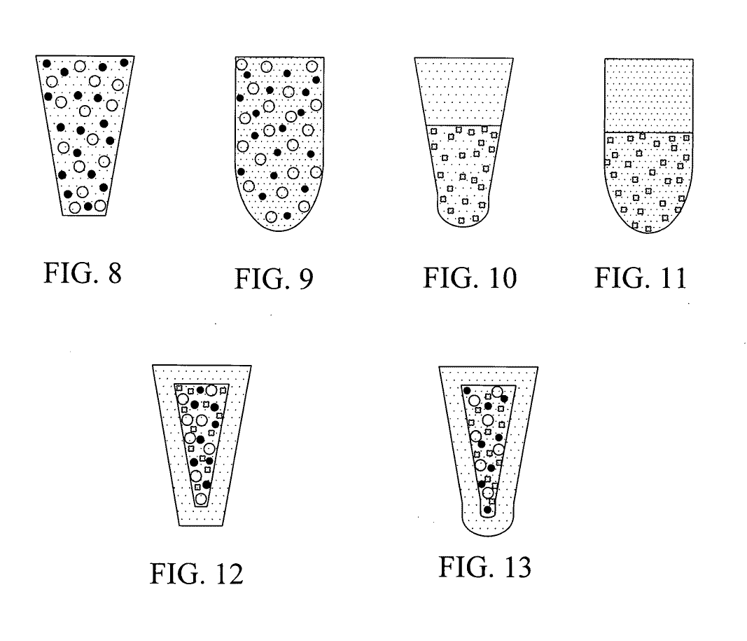

In Examples 5 and 6, biodegradable fillers for restoration of alveolar bones were prepared in the same manner described in Example 1 except the shaping mold 10 of FIG. 1A was replaced by the shaping mold 11 of FIG. 3A and the shaping mold 12 of FIG. 6A, respectively. The fillers prepared in Examples 5 and 6 are shown in FIGS. 8 and 9, respectively.

### Examples 7 and 8

In Examples 7 and 8, biodegradable fillers for restoration of alveolar bones were prepared in the same manner described in Examples 2 or 3 except the shaping mold 11 of FIG. 3A was replaced by the shaping mold 10 of FIG. 1A and the shaping mold 12 of FIG. 6A, respectively. The fillers prepared in Examples 7 and 8 are shown in FIGS. 10 and 11, respectively.

### Examples 9 and 10

In Examples 9 and 10, biodegradable fillers for restoration of alveolar bones were prepared in the same manner described in Example 1 except the shaping mold 12 of FIG. 6A was replaced by the shaping mold 11 of FIG. 3A and the shaping mold 10 of FIG. 1A, respectively. The fillers prepared in Examples 9 and 10 are shown in FIGS. 12 and 13, respectively.

### Comparative Examples 1 to 4

In Comparative Examples 1 to 4, biodegradable fillers for restoration of alveolar bones were prepared in the same manner described in Examples 1 to 4 except the cross-linked collagen fibers used in Examples 1 to 4 was replaced by the non-crosslinked collagen fibers.

### Test Example 1

### Test for Water Absorption Power

First, the fillers prepared in Examples 1 to 4 were precisely weighed by an electronic balance, and the measured weight was the dry weight prior to water absorption. Subsequently, the fillers of Examples 1 to 4 were arranged in a plate with water (10 mL) for 60 seconds, and then taken out for weighing. The absorption powers of the fillers were calculated according the following equation, and the results are shown in the following Table 1.

Water absorption power(%)=[(Wet weight−Dry weight)/Dry weight]*100%

TABLE 1

| | | | Water absorption |
|---|---|---|---|
| Sample | Dry weight (gm) | Wet weight (gm) | power (%) |
| Example 1 | 0.25 | 2.3 | 820 |
| Example 2 | 0.20 | 1.8 | 800 |
| Example 3 | 0.24 | 2.0 | 733 |
| Example 4 | 0.32 | 2.2 | 588 |

According to Table 1, water absorption power of the fillers prepared in Examples 1 to 4 is 5 to 8 times greater than the dry weights thereof.

### Test Example 2

### In Vitro Degradation Test with Collagenase

First, the fillers prepared in Example 1 to 4 and Comparative Example 1 to 4 were used as the samples, of which the size was 0.6 cm (diameter)×1.5 cm (height).

Each sample was degraded in a collagenase solution (10 mL, 0.05 Unit/ml) at 37° C. for 5 days. The samples were taken out at the predetermined time points and observed in regard to their structure. The results are listed in the following Table 2.

TABLE 2

| Test time (Hrs) | 8 | 24 | 48 | 72 | 96 | 120 |
|---|---|---|---|---|---|---|
| Comparative | + | +++ | +++ | +++ | +++ | +++ |
| Example 1 | | | | | | |
| Comparative | + | +++ | +++ | +++ | +++ | +++ |
| Example 2 | | | | | | |
| Comparative | + | +++ | +++ | +++ | +++ | +++ |
| Example 3 | | | | | | |
| Comparative | + | +++ | +++ | +++ | +++ | +++ |
| Example 4 | | | | | | |
| Example 1 | − | + | + | + | ++ | ++ |
| Example 2 | − | + | + | + | ++ | ++ |
| Example 3 | − | + | + | + | ++ | ++ |
| Example 4 | − | + | ++ | ++ | +++ | +++ |

Each group was performed three repeats.
Observation Standard:
− without changes of the appearance
+ degradation to ⅓ volume of the original
++ degradation to ⅓ volume of the original and having no 3D structure
+++ complete degradation

Therefore, the fillers prepared in the examples of the present invention are more complete than those prepared in the comparative examples.

Although the present invention has been explained in relation to its preferred embodiment, it is to be understood that many other possible modifications and variations can be made without departing from the scope of the invention as hereinafter claimed.

What is claimed is:

1. A biodegradable filler for restoration of alveolar bones comprising:
first cross-linked collagen fibers prepared by reacting non-crosslinked collagen fibers with a cross-linking agent; and
supporting particles which are biomedical ceramic particles, bioactive glass, or a combination thereof, and distributed among the first cross-linked collagen fibers.

2. The biodegradable filler as claimed in claim 1, further comprising:
second cross-linked collagen fibers, wherein the first cross-linked collagen fibers and the supporting particles are formed in a first predetermined shape, and the second cross-linked collagen fibers totally encompass the first predetermined shape so as to form a second predetermined shape.

3. The biodegradable filler as claimed in claim 2, wherein the second predetermined shape is a bullet-shaped column, a bulbous-headed cone, or a flat-headed cone.

4. The biodegradable filler as claimed in claim 2, wherein a thickness of the second cross-linked fibers is in a range from 0.1 to 0.3 mm.

5. The biodegradable filler as claimed in claim 2, wherein the first cross-linked collagen fibers and the supporting particles are uniformly distributed in the first predetermined shape.

6. The biodegradable filler as claimed in claim 1, further comprising the second cross-linked fibers, wherein the first cross-linked fibers and the supporting particles are formed in a first predetermined shape, and the second cross-linked fibers are arranged on a surface of the first predetermined shape.

7. The biodegradable filler as claimed in claim 6, wherein a ratio of a thickness of the second cross-linked fibers to a height of the first predetermined shape is in a range from 1:5 to 3:2.

8. The biodegradable filler as claimed in claim 6, wherein the first cross-linked collagen fibers and the supporting particles are uniformly distributed in the first predetermined shape.

9. The biodegradable filler as claimed in claim 1, wherein a particle size of the bioactive glass is in a range from 100 to 700 μm.

10. The biodegradable filler as claimed in claim 1, wherein the biomedical ceramic particles are selected from the group consisting of hydroxyapatite (HAP), β-tri-calcium phosphate (β-TCP), HAP/(3-TCP composite, and a combination thereof.

11. The biodegradable filler as claimed in claim 10, wherein a particle size of the β-tri-calcium phosphate (β-TCP) particles is in a range from 0.5 to 2.0 mm.

12. The biodegradable filler as claimed in claim 10, wherein a particle size of the hydroxyapatite (HAP) particles is in a range from 0.075 to 0.150 mm.

13. The biodegradable filler as claimed in claim 10, wherein a weight ratio of HAP to β-TCP is in a range from 1:1 to 3:1 in the HAP/(3-TCP composite.

14. The biodegradable filler as claimed in claim 1, wherein a weight ratio of the first cross-linked collagen fibers to the supporting particles is in a range from 1:1 to 1:4.

15. The biodegradable filler as claimed in claim 1, wherein the first predetermined shape is a bullet-shaped column, a bulbous-headed cone, or a flat-headed cone.

16. The biodegradable filler as claimed in claim 1, wherein the non-cross-linked collagen fibers are selected from the group consisting of type I collagen, type II collagen, and type III collagen.

17. The biodegradable filler as claimed in claim 1, wherein the cross-linking agent is an aldehyde-based cross-linking agent, a carbodiimide-based cross-linking agent, or a combination thereof.

18. The biodegradable filler as claimed in claim 10, wherein a ratio of the cross-linked collagen fibers to the HAP/β-TCP composite to the bioactive glass is 20-50%:25-40%:25-40% by weight.
